(19) United States
(12) Patent Application Publication
(10) Pub. No.: US 2023/0420111 A1
(43) Pub. Date: Dec. 28, 2023

(54) COMPUTER DEVICE AIDED SELECTION AND ADMINISTRATION OF NEUROHACKS

(71) Applicant: Izzy Justice, Cornelius, NC (US)

(72) Inventor: Izzy Justice, Cornelius, NC (US)

(21) Appl. No.: 18/213,328

(22) Filed: Jun. 23, 2023

Related U.S. Application Data
(60) Provisional application No. 63/355,766, filed on Jun. 27, 2022.

(51) Int. Cl. G16H 20/70; A61B 5/16

(52) U.S. Cl. CPC G16H 20/70; A61B 5/165

(57) ABSTRACT

According to some embodiments, a computing device may be configured to receive user input associated with a user. Based on the user input, the computing device may identify the neurological state of the user. Using the identified neurological state, the computing device may be configured to select a suitable neurohack for administration to the user. The selected neurohack may then administered to the user via the computing device, resulting in an alteration of the neurological state from the identified neurological state to a different neurological state, or reinforcement of the identified neurological state.

### CROSS-REFERENCE TO RELATED APPLICATIONS

The present application claims the benefit of priority to U.S. Provisional Application No. 63/355,766, filed on Jun. 27, 2022, and the entire contents of the above-identified application are incorporated by reference as if set forth herein.

### TECHNICAL FIELD

The present disclosure relates generally to computer systems, methods and computer program products and, more particularly, to computer systems, methods and computer program products for selecting administering neurohacks via computer networks and devices.

### BACKGROUND

Mental health is health. Mental health can affect our physical, psychological, emotional, and social well-being, and mental health disorders can affect our thoughts, feelings, and actions. There is increasing recognition that stress has a large impact on both mental health and physical health.

Stressors may take many forms, and can include environmental, emotional, and physical stressors, each causing some amount of bodily or mental tension in a subject. Although some amount of stress is a normal part of daily life, high amounts of stress and/or long-term stress can become harmful. When stress becomes overwhelming, there is an increase in risk for mental health problems and medical problems.

Effective management of stress and/or a reduction of long-term stress can reduce the occurrence of mental health problems such as depression, anxiety, substance use and abuse, sleep disorders, and pain. Reducing stress can also reduce risk of medical problems such as cardiovascular disease, stroke, sexual dysfunction (e.g., difficulty in copulating or conceiving), headaches, gastrointestinal problems, weakened immune system, high blood pressure, and so on.

Many individuals have experienced significant increases in stress in the past few years (due to various stressors, such as unemployment, climate change, war, famine, and so on). For example, medical professionals and clinicians have experienced extreme increases due in large part to the COVID-19 pandemic, resulting in burnout, fatigue, and emotional exhaustion. This may be a significant contributor to medical errors. The trend is not limited to just medical professions. In the past five years, multiple high-level athletes that have access to a variety of resources have either retired or withdrawn from high-level competition while still in their athletic prime, citing burnout, stress, and mental exhaustion.

Current trends and events suggest that stressors are likely to continue to increase in both number and magnitude, especially as the COVID-19 pandemic continues, and economies of the U.S. and other nations continue to recover and change. Unfortunately, many individuals lack even basic knowledge and awareness of mental health and stress reduction techniques.

### SUMMARY

According to some embodiments, a computing device may be configured to receive user input associated with a user. Based on the user input, the computing device may identify the neurological state of the user. Using the identified neurological state, the computing device may be configured to select a suitable neurohack for administration to the user. The selected neurohack may then administered to the user via the computing device, resulting in an alteration of the neurological state from the identified neurological state to a different neurological state, or reinforcement of the identified neurological state.

According to some embodiments, a non-transitory computer readable media may store instructions for execution by a computing device or a processor thereof. The instructions may result in the computing device being configured to receive user input associated with a user. Based on the user input, the computing device may identify the neurological state of the user. Using the identified neurological state, the computing device may be configured to select a suitable neurohack for administration to the user. The selected neurohack may then administered to the user via the computing device, resulting in an alteration of the neurological state from the identified neurological state to a different neurological state, or reinforcement of the identified neurological state.

### BRIEF DESCRIPTION OF THE DRAWINGS

FIG. 1 is an illustration of an example network useable to administer stress management techniques.

FIG. 2 depicts a schematic of an example user device illustrating components that may engage in administering of stress management techniques.

FIGS. 3, 4, 5, and 6 are flowcharts illustrating operations according to some embodiments of the present disclosure.

FIGS. 7-9 are illustrations of user interface elements that may be used to select and administer neurohacks according to embodiments of the present disclosure

FIG. 10 is a flowchart illustrating operations according to some embodiments of the present disclosure.

Like reference numerals may refer to corresponding parts throughout the figures. Moreover, multiple instances of the same part may be designated by a common prefix separated from an instance number by a dash.

### DETAILED DESCRIPTION

The present disclosure is based in part on the recognition that human bodies, e.g., including various physiological systems and organs of a human, are wired to respond to stressful situations in a variety of ways. For example, the amygdala is a gland designed to release hormones that partially or entirely disable regions of the brain, thereby enabling a person to respond quickly to a perceived danger. The amygdala and other systems and organs in the body have evolved or developed over time as part of the human body's response to physical threats, such as imminent violence, physical danger (e.g., encountering a potential predator), or other situations that may result in bodily harm to a person or a person nearby. Unfortunately, the human body, and the amygdala and other physiological systems and organs, are unable to distinguish between physical threats and emotional threats, and the body is likely to respond to an emotional threat (including stressors at work and at play) in a similar way to its response to a physical threat.

Emotional threats or stressors encountered by a person may create a high alert physiological state, where the brain is operating in a lock down mode. In a sense, the body reacts to high levels of workplace stress in a similar way to encountering a potential source of physical harm (e.g., an oncoming speeding vehicle). The high alert physiological state may result in decreased cognitive performance, as well as result in consequences that affect decision making, such as overgeneralization, becoming more easily aggravated, recalling past negative experiences, struggle to engage in effective social behavior with others, and so on. Physically, the body may react by increasing adrenaline and cortisol, increasing blood flow and heart rate, tensioning muscles for flight or fight, dilating pupils, and so on. If left unchecked or unmanaged, such high alert or high tension states may (and likely will) result in decreased performance, in which a person is unable to respond to emotional stressors, potentially endangering themselves or others due to an inability to think and react clearly and calmly.

When a person is experiencing high physical temperatures (either internally or externally), their body responds by lowering the temperature in an attempt to return to stasis. For example, a high temperature may be responded to by increasing fluid intake, perspiring, seeking shade or indoor environments, and so on. Aspects of the present disclosure provide methods, systems, and devices that assist a user in selecting and administering one or more neurohacks intended to change or alter the neurological state of the user, and in a sense lower their emotional temperature or maintain a low emotional temperature. For example, a user may be in a high emotion neurological state, in which they are experiencing high levels of emotion, such as anger, fear, excitement, love, hate, disgust, frustration, or so on. These emotions may be resultant from a previous or ongoing high stress situation that the user is currently experiencing. The methods, systems, and devices of the present disclosure may help the user in lowering their high emotion neurological state or temperature to a lower level neurological state.

For example, consider an emotional thermometer similar to a traffic light with three readings: red (for high emotional temperature), yellow (for medium emotional temperature), and green (for low or nominal level emotional temperature). When a user is in a green emotional temperature or neurological state, they may be comfortable, happy, stress-free or low-stress, able to think more clearly, and consequently perform at a high level, as the brain is able to retrieve skills and memories that will able good decision-making. When a user is in a green emotional temperature or neurological state, they may experience feels of relaxation, alert senses, and enjoy positive monologues and positive experiences.

Alternatively, when a user is in a yellow emotional temperature or neurological state, they may experience some feelings of stress and/or anxiety. The user may not be as comfortable, happy, or stress-free as in a green emotional temperature or neurological state, and may experience some physical symptoms of stress. Consequently, the ability of the user to perform at a high level may be somewhat degraded, as the brain's ability to retrieve skills and memories that will able good decision-making may be marginally impacted.

At the opposite extreme from a green emotional temperature or neurological state is a red emotional temperature or neurological state. A user may be out of control, filled with and experiencing high levels of emotion, such as anger, fear, excitement, love, hate, disgust, frustration. The user cannot think clearly and cannot engage in good decision-making.

By providing methods, systems, and devices that assist a user in selecting and administering one or more neurohacks intended to change or alter the neurological state of the user, and shift their emotional temperature to a lower value (or maintain an already low emotional temperature), the present disclosure may enable the user to perform or return to performing at a higher level. Moreover, by lowering their neurological temperature or maintaining an already low emotional temperature using the present inventive concepts, a user may be able to engage in more effective management of stress and/or a reduction of long-term stress, and thereby reduce the occurrence of mental health problems such as depression, anxiety, substance use and abuse, sleep disorders, and pain.

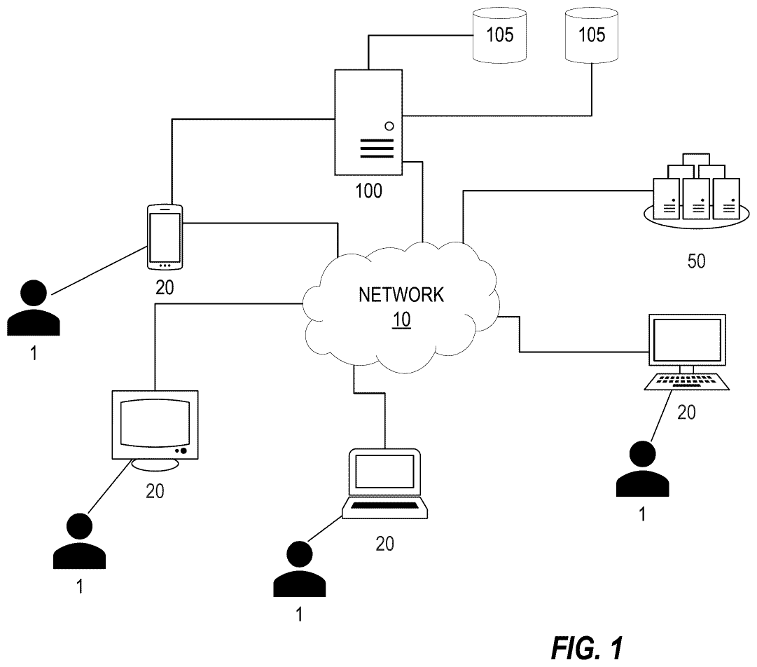

Some embodiments of the present disclosure will now be described with reference to the accompanying drawings. FIG. 1 is an illustration of an example network useable to administer a neurohack. As shown in FIG. 1, a network 10 provides communicative connection among several different user devices 20, and/or to and from users 1 thereof. For example, network 10 could be or include the Internet or another TCP/IP protocol network such as a wide area network (WAN), local area network (LAN), or intranet. In some embodiments, network 10 could be one or more wireless networks each of which implements one or more networking protocols and/or standards, such as Code Division Multiple Access (CDMA), Global System for Mobile Communications (GSM), Long-Term Evolution (LTE), 802.11 standards (Wi-Fi), Bluetooth, Near Field Communications (NFC), etc. Network 10 may meaningful communication between devices 20 and/or other information sources.

One or more providers 50 may connect to one or more user devices 20, either directly or via network 10 or another network. Providers 50 can be any content, media, functionality, software, and/or operations providers for user devices 20. For example, providers 50 may include mobile software developers with server backends and/or access portals for downloading and running software or streaming media on user devices 20. Providers 50 may include a network operator, such as a cellphone and mobile data carrier operating network 10 and controlling access rights of user devices 20 as well as general operation of network 10. Providers 50 may be application storefronts providing search, download, operations connectivity, updating, or the like for applications (sometimes referred to herein in abbreviated form as an app or apps) on devices 20. Providers 50 may be a website or ftp server offering downloadable files or other content that may be displayed or otherwise consumed through user devices 20. Although providers 50 are mostly shown clustered around network 10 for connectivity to user devices 20, it is understood that there may be any direct or indirect connection between any provider 50 and any user device 20.

A user device 20 may be any electronic device, such as a mobile device, capable of performing one or more software-enabled operations, including accepting input from user or from another electronic device and operating based on that input. Examples of user devices 20 may include wearable computers, smartphones, tablets, media players, GPS units, IPTVs, desktop or notebook computers, personal tracking accessories, media and gaming consoles, or the like.

In some embodiments, a user device 20 may be controlled by a user 1 thereof to instantiate an application that performs one or more functions, and/or to resume operation of an application that has been placed in a temporarily suspended state. The application may be configured to communicate with a remote device 100, which may be configured to provide content, data, instructions, or the like responsive to communications transmitted from the application operating on the user device 20. For example, the application may transmit to the remote device 100 a request for data, and the remote device 100 may respond with the data. Such content, data, instructions, or the like may be provided by databases 105 communicatively coupled to the remote device 100. Additionally or alternatively, the remote device 100 may be configured to receive content, data, instructions or the like from the user device 20. Such content, data, instructions, or the like may be communicated responsive to a request transmitted from the remote device 100 (e.g., the remote device 100 may poll the user device 20), or may be communicated in the absence of such a remote device request.

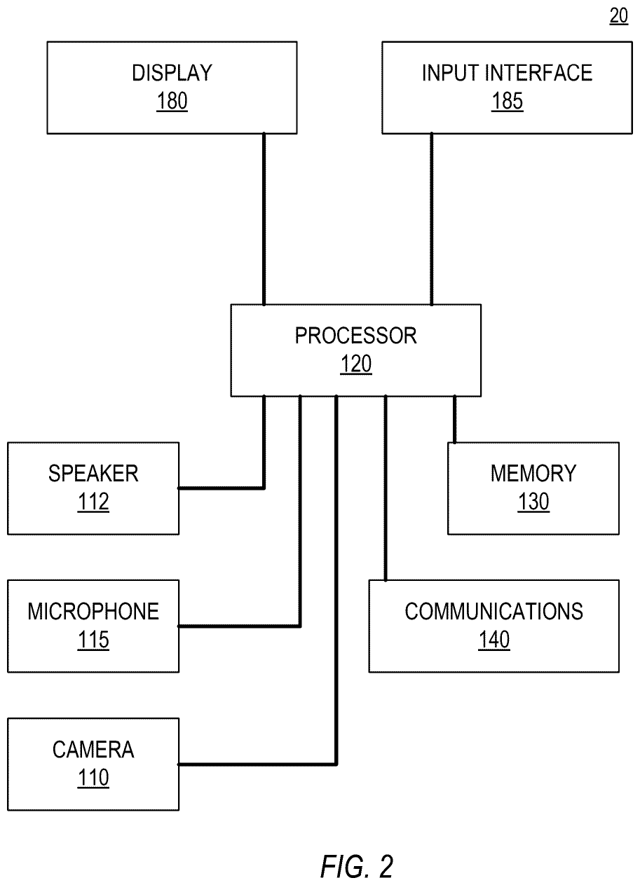

FIG. 2 depicts a schematic of an example user device 20 illustrating components that may engage in selecting and administering of a neurohack. For example, a user device 20 may include a camera 110 including a lens and image sensor, a speaker 112, a microphone 115, a computer processor 120, persistent and/or transient storage 130, display screen 180 (which may comprise a touchscreen or other input reception device or component), input interface 185 for interfacing with one or more input devices, systems, sensors, or the like, and/or communications interface 140. Although elements are shown within a single device 20, it is understood that any element may be separate and connected through appropriate communications such as an external bus or wired or wireless connection.

The processor 120 may include one or more computer processors connected to and specially programmed or otherwise configured to control the various elements of example user device 20. The processor 120 may further be configured to execute example methods, including selecting and administering of a neurohack and reacting in accordance with user input. User input may be received via the input interface 185 (which may include one or more input sensors), the microphone 115, the display screen 180, and/or the camera 110.

The persistent and/or transient storage 130 may be a dedicated data storage drive or device or may be a partition of a general data store in which neurohack data and/or raw data can be saved, stored, located, or the like. The storage 130 may be, for example, random access memory (RAM), read only memory (ROM), programmable read only memory (PROM), erasable programmable read only memory (EPROM), electronically erasable programmable read only memory (EEPROM), flash memory, a hard disk, a processor cache, optical media, other computer readable media, or the like.

The camera 110 may include one or more lenses controlled by lens actuators that move the lenses among different positions to focus the lenses for capturing optical data. Similarly, the camera 110 may adjust focus automatically or in response to user input defining focus locations in the scene being captured. The camera 110 may include image sensor elements such as a charge coupled device (CCD) array, a photodiode array, or any other image sensing device that receives light, potentially via the lens, and generates image data in response to the received light. The camera 110 may be further configured to obtain or adjust image information such as focus, zoom, white balance, exposure, saturation, and/or other image functions. The obtaining or adjusting by the camera 110 may be automatic (e.g. not in response to user input) or responsive to user input received at the user device 20 or other computing device.

The speaker 112 may include any auditory transmission device capable of audio performance of a neurohack or a component thereof. The microphone 115 may include any reception device capable of user input with regard to a neurohack or a component thereof. In some embodiments, the speaker 112 and the microphone 115 may be combined into a single component. In some embodiments, the speaker 112 may be or may include an embedded speaker in a mobile device, and/or the microphone 115 may be or may include an embedded induction microphone in a mobile device.

The display 180 may be a screen, viewfinder, monitor, or any other device capable of visually displaying a neurohack or a component thereof. As discussed above, the display 180 may also comprise an input device or component configured to receive user input, including input received with regard to a neurohack or with regard to selecting a neurohack to be administered. For example, display 180 may be a touchscreen, LCD monitor, CRT monitor, projector, or the like.

The input interface 185 may provide input information with regard to one or more components discussed above, such as the display 180, or with regard to the user device 20 as a whole. For example, if the display 180 is a touchscreen device or other component capable of receiving touch-based input, or the user device 20 is configure to receive touch-based input, input may be received on or via input interface 185 from one or more multi- or single-touch sensors capable of detecting finger or stylus touch, pressure, movement, or the like may be embedded in the display 180 or otherwise located on or within the device 20 to receive the input. These multi- or single-touch sensors may be capacitive sensors. In another example, sensors which may input data via the input interface 185 may include an accelerometer, such as a magnetized compass accelerometer with associated hardware or software or the like, capable of determining device orientation and/or movement. As another example, sensors communicating via the input interface 185 may include a button, keyboard, an external mouse, joystick, or the like and associated hardware or software capable of controlling and determining cursor position and/or activation with respect to the display 180 during operation of the user device 20. The Additional or alternative sensors may be connected to the processor 120, including sensors connected via the input interface 185, and may be capable of deliver sensed input information to the processor 120 including cursor or contact position, duration, numerosity, pressure, movement speed, or the like. For example, sensors may provide communicative data received via a radio frequency (RF) sensor, near-field communication (NFC) sensor, wireless radio transceivers such as Bluetooth or Bluetooth Low Energy or the like, and/or other protocols, systems, or the like. Of course, other examples may be possible.

The processor 120 may be any computer processor, including a processor associated with processor cache, transient memory, video buffer, or the like, specially configured or specially programmed to perform selecting and administering of a neurohack or the same through user device 20. The processor 120 may also be configured or programmed to process any input to device 20, including optical or audio information or signals received via microphone 115 and/or camera 110, including information or signals received as inputs indicative of interaction between a user and a selected and administered neurohack. The processor 120 may also be configured to store or retrieve results, signals, information, data, or the like in the memory 130, including processing instructions stored in a non-transitory format. The processor 120 may also receive sensor information from sensors via an input interface 185 and process the same as an interaction between a user and an administered neurohack. As discussed above, the sensor information may include touch information, cursor information, textual information input via keyboard or other text-entry device, image information, auditory information, or the like. The processor 120 may further include software or configured hardware, or be configured to access, signal, run, operate, and/or execute software or configured hardware, that allows for execution of example methods discussed below.

The user device 20 may further include a communications interface 140 for external wired or wireless communication. For example, communications interface 140 may be an antenna configured to transmit and receive data on radio bands, such as code division multiple access (CDMA) bands, a Wi-Fi antenna, a near field communications (NFC) transmitter/receiver, a GPS receiver, an external serial port or external disk drive or the like. The processor 120 may provide data from the storage 130, data from the camera 110, data received via input interface 185, data from the microphone 115 or the like, to external devices or systems through communications interface 140. Additionally, the user device 20 or devices or components thereof may receive data or signals indicative of data via the communications interface 140, as well as receive neurohack data from one or more providers through communications interface 140. In some aspects, communications interface 140 may function as another input source for user interactions.

Although networked elements and functionalities of example embodiment device 20 are shown in FIG. 2 as individual components with specific groupings and subcomponents, it is understood that these elements may be co-located in a single device having adequately differentiated data storage and/or file systems and processing configurations. Alternatively, the elements shown in FIG. 2 may be remote and plural, with functionality shared across several pieces of hardware, software, or firmware, each communicatively connected to provide data transfer and analysis, if, for example, more resources or better logistics are available in distinct locations. Given the variety of example functions described herein, example user devices 20 may be structured in a variety of ways to provide desired functionality. Other divisions and/or omissions of structures and functionalities among any number of separate modules, processors, and/or servers may be used according to one or more aspects described herein.

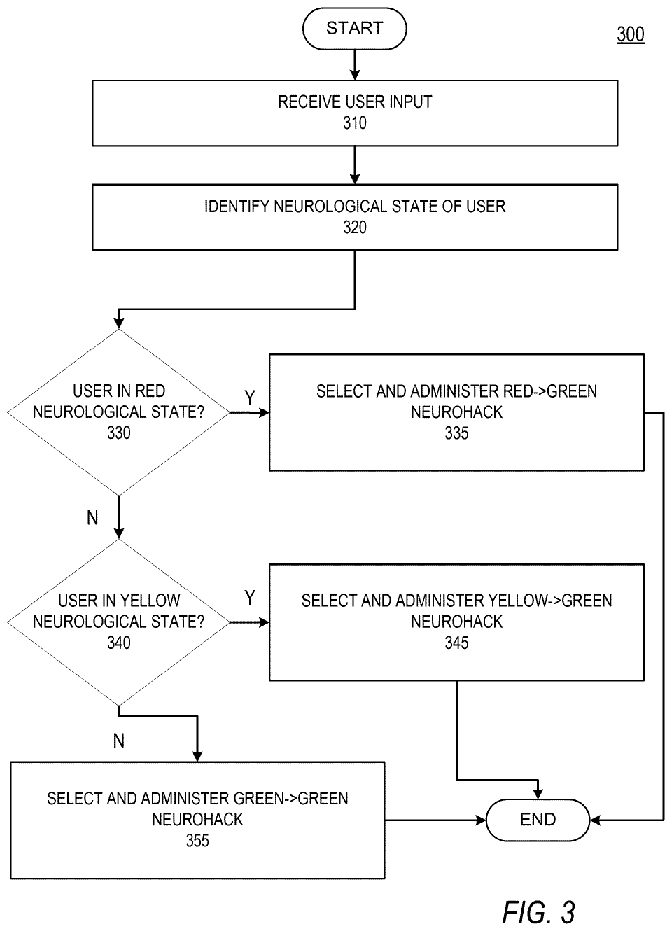

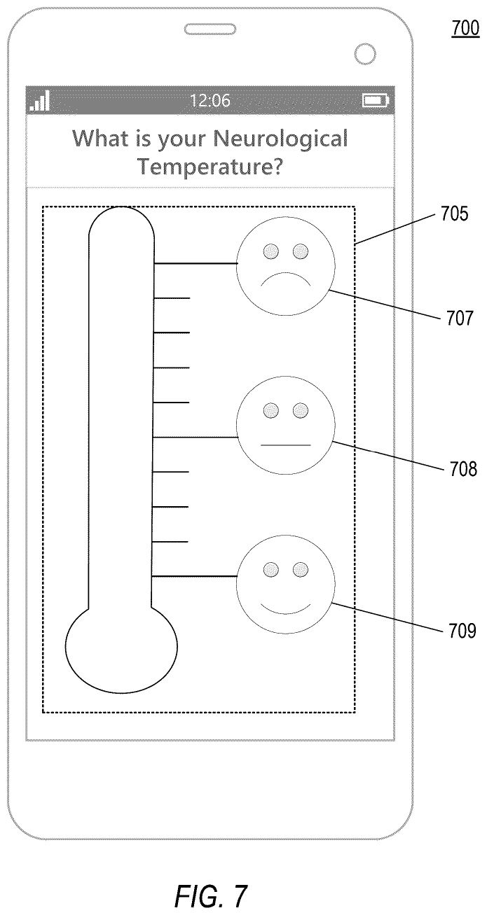

FIGS. 3-6 and 10 are flowcharts illustrating operations according to embodiments of the present disclosure. FIGS. 7-9 are illustrations of user interface elements. More specifically, FIGS. 3-6 and 10 illustrate operations in the selecting and administering of neurohacks according to various embodiments, and FIGS. 7-9 are illustrations of user interface elements that may be used to that may be used to select and administer neurohacks according to embodiments of the present disclosure.

A neurohack may be a mental and/or physical exercise (e.g., a neurological tool) that is intended to alter a person's neurological state and/or emotional temperature. Selection and administration of a neurohack may, in some embodiments, be performed with speed in a relatively brief period of time, e.g., less than five minutes, or even less than one minute. As one example, a neurohack may include a user moving their tongue around their mouth for approximately 20 seconds, slowly feeling the user's teeth, their shape, sizes, edges, and so on. Such an activity may force the brain to process something in the present moment, thereby altering the user's neurological state. As another example, a neurohack may include instructing the user to state three very short and quick positive statements about themselves, another person, or the situation, thereby forcing thinking patterns to shift.

Neurohacks may include:

4-2-8: A user may take a slow breath in, to a count of 4; hold that breath for 2, and then slowly release the breath for a count of 8. Controlled breathing may send neurotransmitters to the brain, thereby lowering intensity of brain waves, which may enable the user to think more clearly.

10-2: A user may grab something with both hands and very quickly squeeze, thereby creating maximum tension in the user's body. This maximum tension may be given a rating of 10. The user may hold the maximum tension for 5 seconds, then slowly lower the tension to a rating of 2. This may reset the tension in the user's body by lowering brain waves, thereby enabling the user to think more clearly.

3Ps (3 Positives): As quickly as possible after something that triggers a negative reaction in a user, the user may state 3 very short and quick positive statements about themself, another person and/or the situation. Examples: I am a good person; I love helping people; I have overcome many challenges. This may enable the brain to shift its thinking patterns, thereby enabling the user to think more clearly.

3S (3 Sounds): The user may close their eyes and listen for three distinctive sounds. Each sound may be focused on to hear different pieces of it. This may force the user to process something in the present moment, relieving it of past/future high brain waves, thereby enabling the user to think more clearly.

3 Eyes: The user may be asked to look at one object wherever they are and find 3 unique attributes about it that were previously unnoticed. This may force the user to process something in the present moment, relieving it of past/future high brain waves, thereby enabling the user to think more clearly.

3F (3 Feelings): The user may close their eyes and identify three unique sensations anywhere in the body or with their hands holding something, feeling the texture of it. Example: Taking a thumb and running down each finger. This may force the user to process something in the present moment, relieving it of past/future high brain waves, thereby enabling the user to think more clearly.

W2-60: The user may whisper to themselves for 60 seconds. The user's self talk may be imagined to be another person talking to the user. The user may then lower the volume, the tone, and/or the intensity of the language. This may enable the brain to shift its thinking patterns, thereby enabling the user to think more clearly.

Tongue 20: The user may close their eyes, and make their tongue slowly feel various mouth features, e.g., teeth, shape, size, edges, etc. for 20 seconds. This may force the user to process something in the present moment, relieving it of past/future high brain waves, thereby enabling the user to think more clearly.

Closed Eyes 30: The user may lean back on a chair/wall where the head can be rested and close their eyes for 30 seconds, thereby allowing the brain to consume other non-visual senses while in that position.

Compass Meditation: This user may follow a sequence similar to that in a compass—North to South, East to West—where North is the top of the head, South is the tips of the toes. East is the tips of the left hand and West is the tips of the right hand.

Traps 10: The user may take one hand and place over the opposite shoulder trapezius (trap) muscle. The user may force down on the trap muscle for 10 seconds, then slowly release with an exhale. This may force the user to process something in the present moment, relieving it of past/future high brain waves, thereby enabling the user to think more clearly.

Hands Roll 10: The user may take a pen/pencil or similar object and place between the hands. The user may gently squeeze the object and move their hands repeatedly over the object, identifying different sensations. This may force the user to process something in the present moment, relieving it of past/future high brain waves, thereby enabling the user to think more clearly.

Hum 30: The user may think of one of their favorite songs or melodies. The user may close their eyes and hum the song or melody as slow as possible. This may force the user to process something known to be enjoyable, relieving it of past/future high brain waves, thereby enabling the user to think more clearly.

Fresh O2: The user may very quickly blow out all the air from their lungs 3 times. The user may imagine wanting “fresh oxygen” in the body. This may force the user to process something in the present moment, relieving it of past/future high brain waves, thereby enabling the user to think more clearly.

Elbow 5-5: The user may take one of their hands and squeeze just below the opposite elbow (thumb on one side and fingers on other side of forearm), gently increasing pressure for 5 seconds and then gently reducing pressure for 5 seconds. This may force the user to process something in the present moment, relieving it of past/future high brain waves, thereby enabling the user to think more clearly.

Wrist 5-5: The user may take one of their hands and squeeze just below the opposite wrist (thumb on one side and fingers on other side of forearm) gently increasing pressure for 5 seconds and then gently reducing pressure for 5 seconds. This may force the user to process something in the present moment, relieving it of past/future high brain waves, thereby enabling the user to think more clearly.

Finger 5-5: The user may take one of their hands and squeeze the tip of any finger on the opposite hand, gently increasing pressure for 5 seconds and then gently reducing pressure for 5 seconds. This may force the user to process something in the present moment, relieving it of past/future high brain waves, thereby enabling the user to think more clearly.

Ear 5-5: The user may take one of their hands and squeeze the bottom tip of the opposite ear (thumb on one side and fingers on other side of ear), gently increasing pressure for 5 seconds and then gently reducing pressure for 5 seconds. This may force the user to process something in the present moment, relieving it of past/future high brain waves, thereby enabling the user to think more clearly.

Knee 5-5: The user may take one of their hands and squeeze just above the knee—same hand on same side knee (thumb on one side and fingers on other side of knee) gently increasing pressure for 5 seconds and then gently reducing pressure for 5 seconds. This may force the user to process something in the present moment, relieving it of past/future high brain waves, thereby enabling the user to think more clearly.

Lobes 5-5: The user may take one of their hands and squeeze just above the area between the eyes and ears (“lobes”) on each side of head. The user may put one thumb on one lobe, palm of hands over face and fingers on the other lobe. The user may gently squeeze increasing pressure for 5 seconds and then gently reducing pressure for 5 seconds. This may force the user to process something in the present moment, relieving it of past/future high brain waves, thereby enabling the user to think more clearly.

Other examples of neurohacks exist and the present disclosure is not necessarily limited to those described herein. In some embodiments, the number of neurohacks may be at least twenty, again with the understanding that the present disclosure is not limited to a specific number of neurohacks.

In FIG. 3 and flowchart 300 thereof, a user device (e.g., user device 20 of FIGS. 1 and 2) may perform a method of selecting and administering a neurohack, which may include receiving user input (block 310). For example, the user may provide their present neurological state or emotional temperature and/or various data that serves as a proxy for the present neurological state or emotional temperature.

As an example, the user may be presented with a user interface 700 seen in FIG. 7, which may include an emotional temperature selection field 705. The emotional temperature selection field 705 may include a red emotional temperature selection item 707, a yellow emotional temperature selection item 708, and a green emotional temperature selection item 709, with the understanding that the present disclosure is not limited thereto, and that different types and numbers of selection items (and selection fields) may be present in a user interface. The user may select one of the red emotional temperature selection item 707, the yellow emotional temperature selection item 708, or the green emotional temperature selection item 709 and thereby provide an indication of their present emotional temperature.

Additionally or alternatively, in some embodiments, various data that serves as a proxy for the user's present neurological state or emotional temperature may be obtained by the user device 20. For example, the user device 20 may include or be communicatively coupled to a sensor or database that can provide a physiological signal (e.g., a heart rate signal, a respiratory signal, a blood pressure signal, or the like), and the user device 20 may obtain the physiological signal from the sensor or database.

Using the received user input, the user device 20 may identify a neurological state of the user (block 320). For example, the user device 20 may identify the selected one of the red emotional temperature selection item 707, the yellow emotional temperature selection item 708, or the green emotional temperature selection item 709. As another example, the user device 20 or other computing device may be configured to convert the proxy information provided from the sensor or database into an identified neurological state.

Using the identified neurological state of the user, an appropriate neurohack may be selected and administered (blocks, 330, 335, 340, 345, 355). For example, the user may be determined to be in a red emotional temperature or neurological state or (Y branch from block 330), and an appropriate neurohack that is intended to reduce the emotional temperature or neurological state of the user from red to green may be selected and administered (block 335). As a second example, the user may be determined to be in a yellow emotional temperature or neurological state or (N branch from block 330, Y branch from block 340), and an appropriate neurohack that is intended to reduce the emotional temperature or neurological state of the user from yellow to green may be selected and administered (block 345). As a third example, the user may be determined to be in a green emotional temperature or neurological state or (N branch from block 330, N branch from block 340), and an appropriate neurohack that is intended to maintain the green emotional temperature or neurological state of the user may be selected and administered (block 355).

Although one set of operations to select and administer an appropriate neurohack is shown in FIG. 3, it is understood that different algorithms may be implemented to arrive at the same result. For example, a switch/case statement may be used, a different series of conditions may be used (e.g., checking first for green, then yellow, then red, or any other order), and so on.

In some embodiments, selecting and administering an appropriate neurohack (blocks 335, 345, and 355) may include accessing a list or database of neurohacks. The list or database of neurohacks may include tagged information indicating whether a particular neurohack is a red->green neurohack (selectable in block 335), a yellow->green neurohack (selectable in block 345), and/or a green->green neurohack (selectable in block 355). In some embodiments, segmented or separate lists may be maintained for each emotional temperature or neurological state.

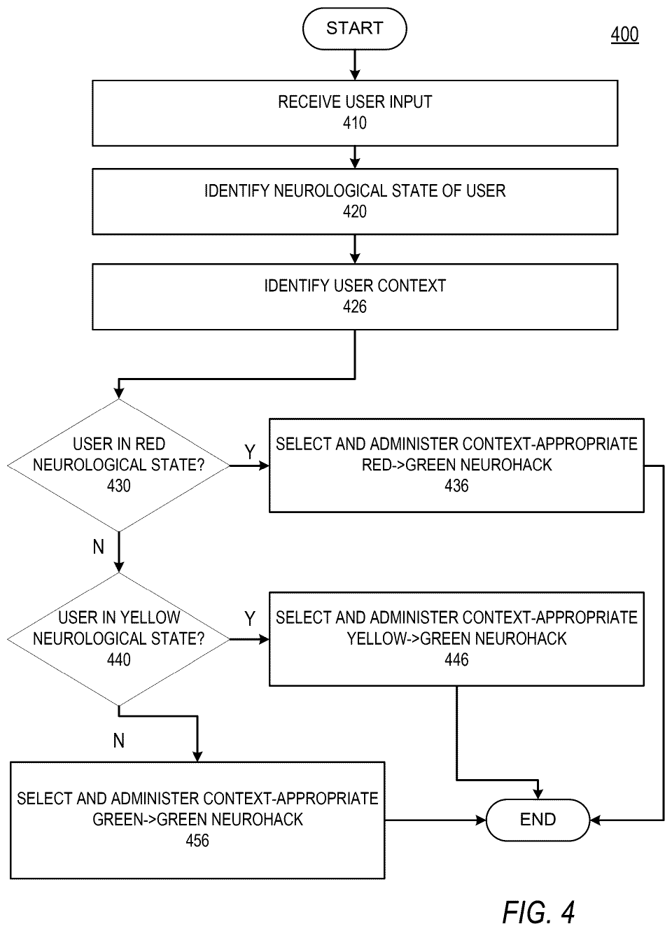

In some embodiments, and with reference to FIG. 4, neurohacks may be selected as appropriate for a present context for the user. For example, a user may be in a location or situation (e.g., on their commute to or from work, at home, at school, at work, on vacation, at a party, engaging in recreation, etc.) where some neurohacks may be more appropriate or less appropriate. A neurohack in which a user must verbally identify nearby objects, for example, may be less appropriate at work than a neurohack in which the user may remain silent. As another example, if the user is operating a motor vehicle, a first neurohack in which the user must move their feet or other body part may be less appropriate than a second neurohack where the user may remain stationary or physically engaged in operating the motor vehicle. As yet another example, some neurohacks may be targeted to be used just before the user goes to bed (for a short nap or a long sleep). Such neurohacks may target a natural functionality of the brain to prioritize negative experiences of the day for storage. Using one or more neurohacks before sleeping may enable a user to better balance storage of experiences, thereby enhancing quality of sleep and/or enabling the brain to be at lower frequencies for the next day or when awakening from sleep.

Accordingly, in FIG. 4 and flowchart 400 thereof, a user device (e.g., user device 20 of FIGS. 1 and 2) may perform a method of selecting and administering a context-appropriate neurohack. Aspects of blocks 410, 420, 430, and 440 of FIG. 4 may be similar to blocks 310, 320, 330, and 340 of FIG. 3, and reference is therefore made to the discussion of the similar blocks above.

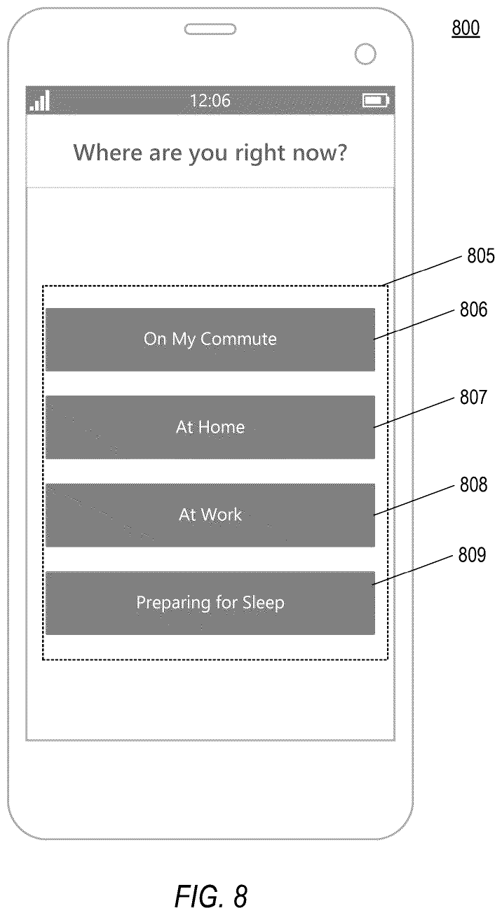

Additionally, as part of receiving user input (block 410), the user may provide their present context and/or various data may be obtained that serves as a proxy for the present context of the user. As an example, the user may be presented with a user interface 800 seen in FIG. 8, which may include a context selection field 805. The context selection field 805 may include various contexts, such as a commute context selection item 806, an at home context selection item 807, at work context selection item 808, and a sleep preparation context selection item 809. The present disclosure is not limited to the examples of contexts illustrated in FIG. 8, and different types and numbers of selection items (and selection fields) may be present in a user interface 800. The user may select one of the commute context selection item 806, the at home context selection item 807, the work context selection item 808, and the sleep preparation context selection item 809 and thereby provide an indication of their present context.

Additionally or alternatively, various data that serves as a proxy for the user's present context may be obtained by the user device 20. For example, the user device 20 may include or be communicatively coupled to a sensor or database that can provide a geolocation signal identifying a geolocation of the user, and the user device 20 may obtain the physiological signal from the sensor or database. As another example, active low-power wireless connections between the user device 20 and other devices (indicating the devices are in proximity to one another) may be used to identify a context of the user.

Using the received user input and/or context proxy, the user device 20 may identify a context of the user (block 426). For example, the user device 20 may identify the selected one of the commute context selection item 806, the at home context selection item 807, the work context selection item 808, and the sleep preparation context selection item 809.

Using the identified neurological state and context of the user, a context-appropriate neurohack may be selected and administered (blocks, 430, 436, 440, 445, 456). For example, the user may be determined to be in a red emotional temperature or neurological state or (Y branch from block 430), and a context-appropriate neurohack that is intended to reduce the emotional temperature or neurological state of the user from red to green may be selected and administered (block 436). As a second example, the user may be determined to be in a yellow emotional temperature or neurological state or (N branch from block 430, Y branch from block 440), and a context-appropriate neurohack that is intended to reduce the emotional temperature or neurological state of the user from yellow to green may be selected and administered (block 446). As a third example, the user may be determined to be in a green emotional temperature or neurological state or (N branch from block 430, N branch from block 440), and a context-appropriate neurohack that is intended to maintain the green emotional temperature or neurological state of the user may be selected and administered (block 456).

As with FIG. 3, although one set of operations to select and administer a context appropriate neurohack is shown in FIG. 4, it is understood that different algorithms may be implemented to arrive at the same result. For example, a switch/case statement may be used, a different series of conditions may be used (e.g., checking first for green, then yellow, then red, or any other order), and so on.

In some embodiments, selecting and administering a context appropriate neurohack (blocks 436, 446, and 456) may include accessing a list or database of neurohacks. The list or database of neurohacks may include tagged information indicating whether a particular neurohack is a red->green neurohack (selectable in block 436), a yellow->green neurohack (selectable in block 446), and/or a green->green neurohack (selectable in block 456). The tagged information may also indicate one or more contexts for which the neurohack may be selected. In some embodiments, segmented or separate lists may be maintained for each context and emotional temperature or neurological state.

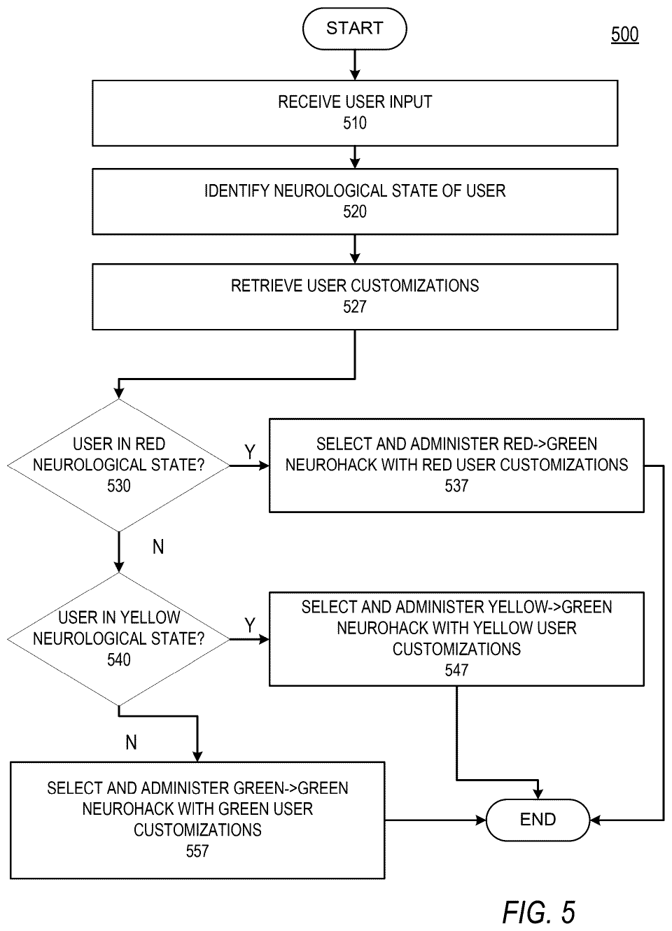

In some embodiments, and with reference to FIG. 5, neurohacks may be customized as appropriate for a present user. For example, a user may experience a greater degree of shift in neurological state or emotional temperature if presented with a still image or video (sequence of images, with or without audio) of a favorite location or family member, text recalling a favorite memory or food, favorite music or other audio (e.g., sound effect, spoken quote, or the like), and/or other personalized information.

Accordingly, in FIG. 5 and flowchart 500 thereof, a user device (e.g., user device 20 of FIGS. 1 and 2) may perform a method of selecting and administering a customized neurohack. Aspects of blocks 510, 520, 530, and 540 of FIG. 5 may be similar to blocks 310, 320, 330, and 340 of FIG. 3, and reference is therefore made to the discussion of the similar blocks above.

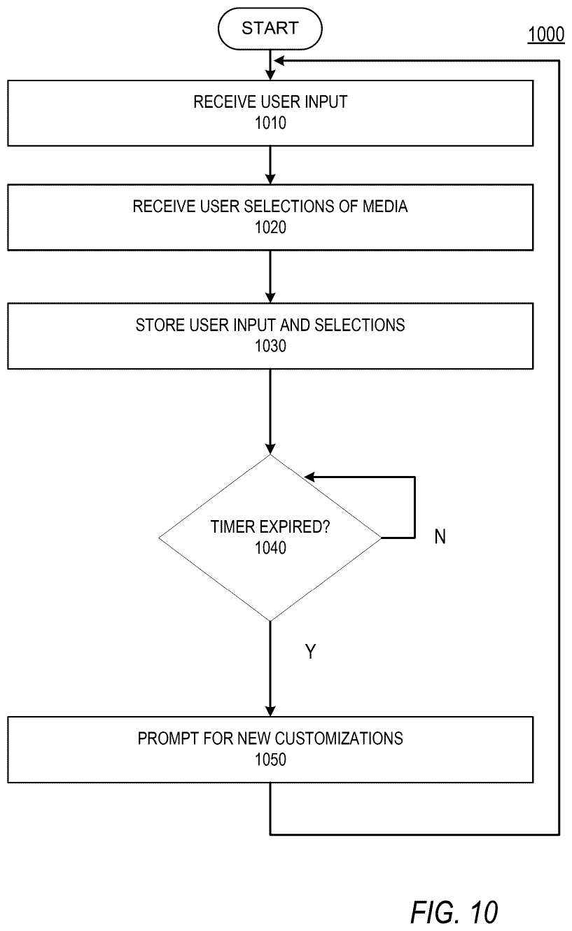

Additionally, the user may provide customization information. This may be performed once during instantiation or installation of a mobile application that performs the method of FIG. 5, or periodically. For example, as seen in FIG. 10, the user may provide user input and/or selections of media stored locally on the user device 20 (blocks 1010, 1020). In some embodiments, the user may be provided with various prompts and/or entry fields in which customization information may be provided or selected (e.g., an image picker, media file picker, or the like). The provided information may be stored (block 1030). The provided information may be stored locally, thereby improving security as the selections are not passed via a network. In some embodiments, various data that serves as a proxy for customizations appropriate for the user may be obtained by the user device 20. For example, the user device 20 may include or be communicatively coupled to a sensor or database that can provide a signal identifying favorite or frequently-experienced music, foods, locations, video files, audio files, image files, text files, websites, telephone or social media contacts accessed by the user, and the user device 20 may obtain the signal from the sensor or database. Periodically, e.g., after expiration of a timer (“Y” branch from block 1040), the user may be prompted for new customizations (block 1050) to maintain a state of freshness of the provided information.

Returning to FIG. 5, the user device 20 may retrieve the customizations for the user (block 527), and use the retrieved customizations to modify the selected neurohacks (blocks 537, 547, 557). For example, using the identified neurological state and customizations of the user, a customized neurohack may be selected and administered. The user may be determined to be in a red emotional temperature or neurological state or (Y branch from block 530), and a customized neurohack that is intended to reduce the emotional temperature or neurological state of the user from red to green may be selected and administered (block 537). As a second example, the user may be determined to be in a yellow emotional temperature or neurological state or (N branch from block 530, Y branch from block 540), and a customized neurohack that is intended to reduce the emotional temperature or neurological state of the user from yellow to green may be selected and administered (block 547). As a third example, the user may be determined to be in a green emotional temperature or neurological state or (N branch from block 530, N branch from block 540), and a customized neurohack that is intended to maintain the green emotional temperature or neurological state of the user may be selected and administered (block 557).

As with FIG. 3, although one set of operations to select and administer a customized neurohack is shown in FIG. 5, it is understood that different algorithms may be implemented to arrive at the same result. For example, a switch/case statement may be used, a different series of conditions may be used (e.g., checking first for green, then yellow, then red, or any other order), and so on.

In some embodiments, selecting and administering a customized neurohack (blocks 537, 547, and 557) may include accessing a list or database of neurohacks. The list or database of neurohacks may include tagged information indicating whether a particular neurohack is a red->green neurohack (selectable in block 537), a yellow->green neurohack (selectable in block 547), and/or a green->green neurohack (selectable in block 557). The tagged information may also indicate one or more customizations and may indicate which customizations are appropriate for each neurohack and/or neurological state. In some embodiments, segmented or separate lists of customizations may be maintained for each emotional temperature or neurological state.

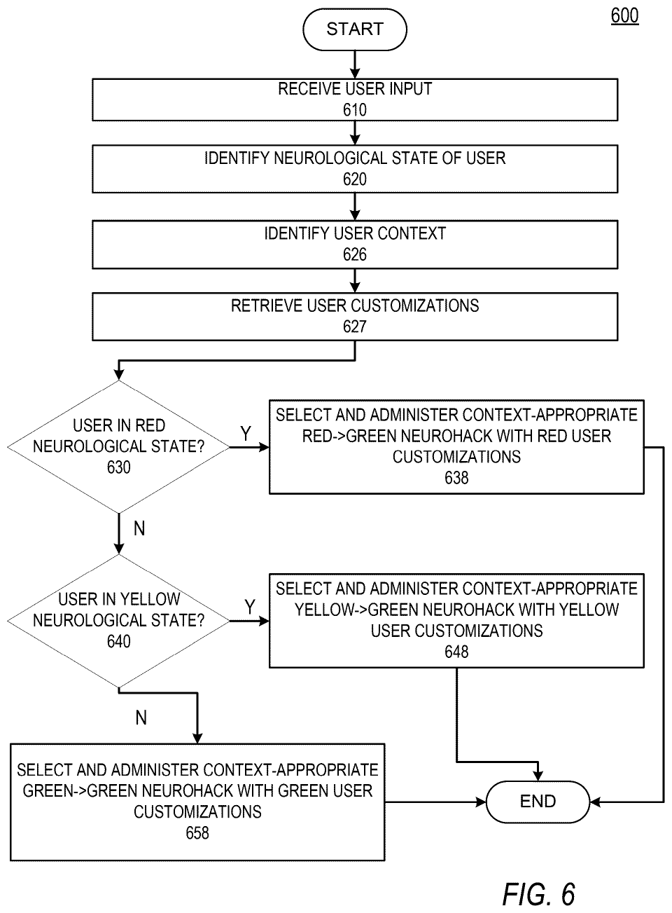

In some embodiments, and with reference to FIG. 6 and flowchart 600 thereof, neurohacks may be customized and context-appropriate for a present user. For example, a user may experience an even larger degree of shift in neurological state or emotional temperature if presented with an image of a favorite location or family member, text recalling a favorite memory or food, favorite music, and/or other personalized information and able to administer the neurohack in a context-appropriate manner.

Accordingly, in FIG. 6 and flowchart 600 thereof, a user device (e.g., user device 20 of FIGS. 1 and 2) may perform a method of selecting and administering a customized and context-appropriate neurohack. Aspects of blocks 610, 620, 630, and 640 of FIG. 6 may be similar to blocks 310, 320, 330, and 340 of FIG. 3, and reference is therefore made to the discussion of the similar blocks above. Aspects of blocks 626 and 627 may be respectively similar to blocks 426 of FIG. 4 and 527 of FIG. 5, and reference is therefore made to the discussion of the similar blocks above.

The user device 20 may retrieve the customizations for the user (block 627), and use the retrieved customizations to modify a context-appropriate selected neurohack (blocks 638, 648, 658). For example, using the identified neurological state and customizations of the user, a customized context-appropriate neurohack may be selected and administered. The user may be determined to be in a red emotional temperature or neurological state or (Y branch from block 630), and a customized and context-appropriate neurohack that is intended to reduce the emotional temperature or neurological state of the user from red to green may be selected and administered (block 638). As a second example, the user may be determined to be in a yellow emotional temperature or neurological state or (N branch from block 630, Y branch from block 640), and a customized and context-appropriate neurohack that is intended to reduce the emotional temperature or neurological state of the user from yellow to green may be selected and administered (block 648). As a third example, the user may be determined to be in a green emotional temperature or neurological state or (N branch from block 630, N branch from block 640), and a customized and context-appropriate neurohack that is intended to maintain the green emotional temperature or neurological state of the user may be selected and administered (block 658).

As with FIG. 3, although one set of operations to select and administer a context appropriate neurohack is shown in FIG. 5, it is understood that different algorithms may be implemented to arrive at the same result. For example, a switch/case statement may be used, a different series of conditions may be used (e.g., checking first for green, then yellow, then red, or any other order), and so on.

In some embodiments, selecting and administering a customized and context-appropriate neurohack (blocks 638, 648, and 658) may include accessing a list or database of neurohacks. The list or database of neurohacks may include tagged information indicating whether a particular neurohack is a red->green neurohack (selectable in block 537), a yellow->green neurohack (selectable in block 547), and/or a green->green neurohack (selectable in block 557). The tagged information may also indicate one or more customizations and may indicate which customizations are appropriate for each neurohack and/or neurological state. The tagged information may also indicate one or more contexts and may indicate which contexts are appropriate for each neurohack and/or neurological state. In some embodiments, segmented or separate lists of customizations and/or contexts may be maintained for each emotional temperature or neurological state.

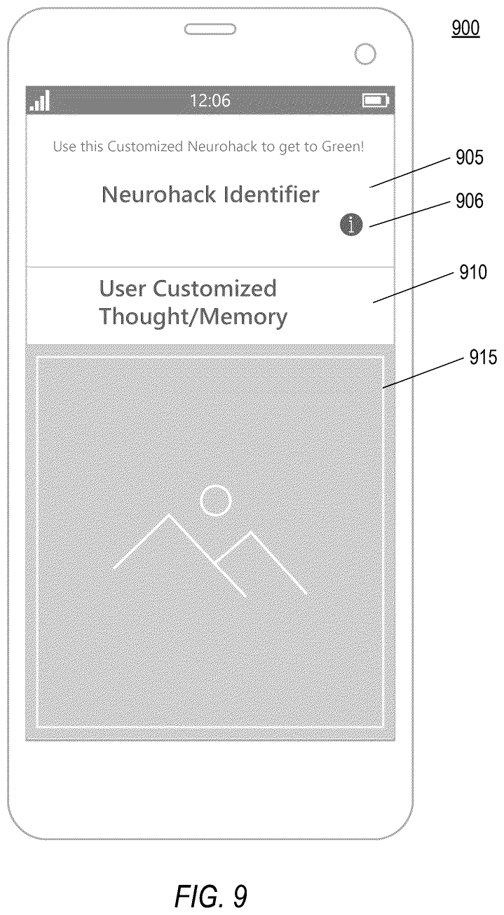

FIG. 9 illustrates an example user interface 900 indicating a customized neurohack to be administered to a user. The customized neurohack user interface 900 may include a neurohack identifier 905, assistance icon 906, user customized text field 910, and user customized image field 915. The neurohack identifier 905 may indicate the selected neurohack (e.g., selected via the flowchart of FIG. 5 or FIG. 6), and the assistance icon 906 may be selectable by a user to receive additional information or instructions regarding the neurohack. The user customized text field 910 and user customized image field 915 may indicate textual and/or pictorial information to user to enhance a degree of shift in the emotional temperature of the user.

Embodiments of the present inventive concepts have been described above with reference to the accompanying drawings, in which embodiments of the inventive concepts are shown. The inventive concepts may, however, be embodied in many different forms and should not be construed as limited to the embodiments set forth herein. Rather, these embodiments are provided so that this disclosure will be thorough and complete, and will fully convey the scope of the inventive concepts to those skilled in the art. Like numbers refer to like elements throughout.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present inventive concepts. As used herein, the term “and/or” includes any and all combinations of one or more of the associated listed items.

It will be understood that when an element is referred to as being “on” another element, it can be directly on the other element or intervening elements may also be present. In contrast, when an element is referred to as being “directly on” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between”, “adjacent” versus “directly adjacent”, etc.).

Relative terms such as “below” or “above” or “upper” or “lower” or “horizontal” or “vertical” may be used herein to describe a relationship of one element, layer or region to another element, layer or region as illustrated in the figures. It will be understood that these terms are intended to encompass different orientations of the device in addition to the orientation depicted in the figures.

The terminology used herein is for the purpose of describing particular embodiments only and is not intended to be limiting of the inventive concepts. As used herein, the singular forms “a”, “an” and “the” are intended to include the plural forms as well, unless the context clearly indicates otherwise. It will be further understood that the terms “comprises” “comprising,” “includes” and/or “including” when used herein, specify the presence of stated features, operations, elements, and/or components, but do not preclude the presence or addition of one or more other features, operations, elements, components, and/or groups thereof.

Aspects and elements of all of the embodiments disclosed above can be combined in any way and/or combination with aspects or elements of other embodiments to provide a plurality of additional embodiments.

What is claimed is:

1. A method comprising:
receiving, by a computing device, user input associated with a user;
identifying, based on the user input, a neurological state of the user; and
selecting a neurohack for administration to the user based on the identified neurological state of the user, and
administering the selected neurohack to the user, thereby altering the neurological state of the user from the identified neurological state to a different neurological state or reinforcing the identified neurological state.

2. The method of claim 1, further comprising identifying a present context of the user, wherein selecting the neurohack for administration to the user based on the identified neurological state of the user comprises selecting a context-appropriate neurohack based on the identified present context of the user.

3. The method of claim 2, further comprising receiving a location of the user, wherein the identifying of the present context of the user is based on the received location of the user.

4. The method of claim 1, wherein administering the selected neurohack to the user comprises displaying the selected neurohack to the user via a user interface.

5. The method of claim 4, wherein the selected neurohack to the user is displayed in the user interface with instructions indicating how the selected neurohack is to be performed.

6. The method of claim 1, further comprising:
retrieving neurohack customizations associated with the user; and
modifying the selected neurohack for administration based on the retrieved neurohack customizations, resulting in a customized neurohack.
wherein administering the selected neurohack to the user comprises administering the selected neurohack.

7. The method of claim 6, wherein the retrieved neurohack customizations comprise an image, and wherein administering the customized neurohack comprises displaying the image to the user via a user interface.

8. The method of claim 1, wherein identifying the neurological state of the user comprises receiving data that serves as a proxy for the present neurological state of the user.

9. A non-transitory computer-readable storage medium having stored thereon instructions that, when executed by a processor, cause the processor to:
receive user input associated with a user;
identify, based on the user input, a neurological state of the user;
select a neurohack for administration to the user based on the identified neurological state of the user; and
administer the selected neurohack to the user using the computing device, thereby altering the neurological state of the user from the identified neurological state to a different neurological state or reinforcing the identified neurological state of the user.

10. The non-transitory computer-readable storage medium of claim 9, having stored thereon further instructions that, when executed by the processor, cause the processor to:
identify a present context of the user, wherein selection of the neurohack based on the identified neurological state of the user comprises selection of a context-appropriate neurohack based on the identified present context of the user.

11. The non-transitory computer-readable storage medium of claim 10, having stored thereon further instructions that, when executed by the processor, cause the processor to:
receive a location of the user, wherein identification of the present context of the user is based on the received location of the user.

12. The non-transitory computer-readable storage medium of claim 9, having stored thereon further instructions that, when executed by the processor, cause the processor to administer the selected neurohack to the user by displaying the selected neurohack to the user via a user interface.

13. The non-transitory computer-readable storage medium of claim 12, having stored thereon further instructions that, when executed by the processor, cause the processor to display the selected neurohack to the user with instructions indicating how the selected neurohack is to be performed.

14. The non-transitory computer-readable storage medium of claim 9, having stored thereon further instructions that, when executed by the processor, cause the processor to:
retrieve neurohack customizations associated with the user; and
modify the selected neurohack for administration based on the retrieved neurohack customizations, resulting in a customized neurohack.
wherein administration of the selected neurohack to the user comprises administration of the selected neurohack.

15. The non-transitory computer-readable storage medium of claim 14, wherein the retrieved neurohack customizations comprise an image, and wherein administration of the customized neurohack comprises displaying the image to the user via a user interface.

16. An apparatus comprising:
a processor; and
memory storing non-transitory computer-readable instructions that, when executed by the processor, cause the processor to:
receive user input associated with a user,
identify, based on the received user input, a neurological state of the user,
select a neurohack for administration to the user based on the identified neurological state of the user, and
administer the selected neurohack to the user.

17. The apparatus of claim 16, wherein the memory stores further non-transitory computer-readable instructions that cause the processor to identify a present context of the user, wherein selection of the neurohack based on the identified neurological state of the user comprises selection of a context-appropriate neurohack based on the identified present context of the user.

18. The apparatus of claim 16, further comprising a display device, wherein the instructions that, when executed by the processor, cause the processor to administer the selected neurohack to the user comprise instructions that cause the processor to display the selected neurohack to the user via the display device.

19. The apparatus of claim 16, wherein the memory stores further non-transitory computer-readable instructions that, when executed by the processor, cause the processor to display the selected neurohack to the user with instructions indicating how the selected neurohack is to be performed.

20. The apparatus of claim 16, wherein the memory stores further non-transitory computer-readable instructions that, when executed by the processor, cause the processor to:
retrieve neurohack customizations associated with the user; and
modify the selected neurohack for administration based on the retrieved neurohack customizations, resulting in a customized neurohack.
wherein administration of the selected neurohack to the user comprises administration of the selected neurohack.
